(19) United States
(12) Patent Application Publication
(10) Pub. No.: US 2022/0068488 A1
(43) Pub. Date: Mar. 3, 2022

(54) DATA PROCESSING SYSTEM AND METHOD FOR DETERMINING THE RISK OF A TRANSFER OF AN INDIVIDUAL TO THE EMERGENCY DEPARTMENT

(71) Applicant: JACQUES-HENRI VEYRON, NEUILLY-SUR-SEINE (FR)

(72) Inventor: JACQUES-HENRI VEYRON, NEUILLY-SUR-SEINE (FR)

(21) Appl. No.: 17/298,915

(22) PCT Filed: Nov. 18, 2019
(86) PCT No.: PCT/EP2019/081658

§ 371 Date: Jun. 1, 2021

(30) Foreign Application Priority Data
Nov. 29, 2018 (FR) 1872055

(51) Int. Cl. G16H 50/30; G16H 10/60; G16H 50/20

(52) U.S. Cl. CPC G16H 50/30; G16H 50/20; G16H 10/60

(57) ABSTRACT

A data processing system to determine a risk factor of an impending transfer to the emergency department of an individual. The system includes a database which stores a plurality of status records for each person of a set of people. Each status record is dated and includes a list of monitoring indicators. Each indicator has a value chosen from a list of two predetermined values according to the status of the corresponding person. The system also a microprocessor to determine the risk factor by analyzing a plurality of status records of the individual, filled out according to the status of the individual, using a machine learning algorithm. The parameters of the algorithm being generated beforehand. A data processing method is further provided to determine a risk factor for an impending transfer of the individual to the emergency department.

### TECHNICAL FIELD OF THE INVENTION

The field of the invention is the computer field.

More specifically, the invention relates to a data processing system and method for determining the risk of a transfer of an individual to the emergency department.

In particular, the invention finds applications in the field of monitoring persons, more specifically elderly people, living autonomously at their home, generally non-medicalized. In general, the persons monitored by the system of the present invention have several pathologies.

### BACKGROUND OF THE INVENTION

Techniques for remote monitoring of the condition of an individual are known from the prior art.

In general, such techniques are based on the use of sensors measuring at least one physiological data of the individual, amongst the heart rate, the blood pressure, the temperature, the oxygen or glucose level in the blood, etc.

The major drawback of these techniques is that they require a regular recording of these physiological data for the processing of the data to be reliable to determine the condition of the individual.

This regular recording may further turn out to be very binding for the individual, and even requiring a regular intervention of a medical attendant to perform more technical acts, such as a blood sample analyzed subsequently.

Moreover, the monitoring of a population at risk turns out to be tedious for healthcare professionals who have to analyze the physiological data of a large number of individuals.

To facilitate the work of healthcare professionals, automatic data processing techniques have been suggested, based in particular on statistical analyses of physiological data on a large scale.

In general, such techniques are dedicated to the prediction of a particular pathology and consequently turn out to be unreliable to determine a risk of admission of an individual having a multitude of pathologies at once to the emergency department.

None of the current systems does allow addressing all of the required needs simultaneously, namely providing a technique for determining, more efficiently, a risk of transfer of an individual having several pathologies at once to the emergency department, in the near future within seven days, that is reliable and merely binding for the individual.

### OBJECT AND SUMMARY OF THE INVENTION

These objectives, as well as other ones that will come out only later on, are achieved using a data processing system for determining a risk factor of an imminent transfer of an individual to the emergency department.

The objective of the present invention is to enable predicting a transfer of the individual to the emergency department within the next seven days with a predictive performance higher than 50%, preferably at least 65%, more preferably higher than 70%.

In general, such a system comprises a computer server provided with a microprocessor and a computer memory.

According to the invention, the data processing system also comprises:
a database storing a plurality of status sheets for each person of a group of persons and a database storing the dates of transfers of said group of persons to the emergency department, each status sheet being dated and including a list of monitoring indicators, each indicator having a value selected in a list of two predetermined values according to the status of the corresponding person;
means for generating the parameters of an automatic learning computer algorithm from the status sheets and the dates of transfers of the group of persons to the emergency department;
means for filling a status sheet of the individual, the status sheet including the list of monitoring indicators, each indicator having a value selected in a list of two predetermined values according to the status of the individual;
means for determining the risk factor by analysis of a plurality of status sheets of the individual thanks to the automatic learning algorithm whose parameters have been generated beforehand, the status sheets of the individual being established at distinct time points; and
means for generating a warning when the risk factor exceeds a predetermined threshold.

Thus, it is possible to predict a risk of transfer to the emergency department within an imminent time period, generally within the next seven days.

It should be highlighted that the determination of the risk factor is performed without any analysis of the physiological data of the individual, these not being included in the status sheet. Moreover, the value of the risk factor does not provide any indication with regards to a pathology of the individual.

In general, the status sheet comprises a plurality of monitoring indicators whose values may be determined by the individual, by an assistant or by a companion, without requiring any prior medical knowledge. In general, the possible values consist of a positive value (for example: “Yes”) and a negative value (for example: “No”). In other words, these monitoring indicators are determined according to an observation of the individual.

Thus, the monitoring of the condition of the person is simple to implement and merely binding.

Furthermore, the parameters generated from a very large number of status sheets and of transfers to the emergency department recorded beforehand allow determining the risk factor for the individual by analyzing the evolution of the status sheets recorded on a regular basis, for example every week or two to three times a week.

It should be highlighted that the monitored individual is generally an elderly person rarely having only one pathology but several pathologies at once, which increases the risk factor of transfer to the emergency department.

By using indicators having a limited number of possible values, it is thus possible to deduce, thanks to a large-scale analysis, a risk factor of transfer of the individual to the emergency department.

The warning may be in the form of text such as a message intended for a practitioner, in the form of light and/or in the form of sound, who, consequently, can monitor the condition of the individual without having to make regular trips. For example, the predetermined threshold may be in the range of 40%, 50% or 60%.

A risk indicator may also be determined according to the risk factor to indicate whether the risk is considerable, whether vigilance is needed or whether the risk is low.

It should also be highlighted that the invention is implemented by a computer allowing processing a very large number of data, in general greater than a few tens of data, in a short period of time. This automatic processing allows determining the parameters of the automatic learning computer algorithm that will be used for the determination of the risk factor of a transfer to the emergency department in the near future.

Advantageously, the monitoring indicators of the list of each status sheet are selected among:
indicators related to the health condition of the individual, such as:
   A1. the individual has swollen legs;
   A2. the individual has difficulties in breathing;
   A3. the individual is feverish;
   A4. the individual has pains;
relational-type indictors, such as:
   B1. the individual is indifferent;
   B2. the individual is not very communicative;
   B3. the individual lives alone since at least seven days;
   B4. the individual has contacts or visits with his entourage;
behavioral-type indicators, such as:
   C1. the individual refuses toileting assistance;
   C2. the individual does not recognize the companion;
   C3. the individual forgets when the companion has come by;
   C4. the individual communicates inconsistently;
   C5. the individual is aggressive;
   C6. the individual is sad;
   C7. the individual stores objects in inappropriate locations;
   C8. the individual seems tired;
   C9. the individual refuses the intervention of the companion;
indicators representative of the physical and sensory capabilities of the individual, such as:
   D1. the individual stands up;
   D2. the individual moves at his home;
   D3. the individual washes himself;
   D4. the individual prepares his meals;
   D5. the individual leaves his home;
   D6. the individual eats;
   D7. the individual falls.

These indicators may be accompanied with indicators relating to the assistant such as:
E1. the assistant is sad;
E2. the assistant is exhausted.

Advantageously, all or part of the monitoring indicators are associated to a sub-indicator indicating the evolution of said indictor in comparison with the last filling of the status sheet. The sub-indicator is selected amongst three values generally corresponding to an improvement of the status, to a stabilization of the status and to a degradation of the status.

Preferably, the list of the monitoring indicators of each status sheet comprises at least four monitoring indicators.

In other words, the data analysis for determining the risk factor of a transfer to the emergency department is performed on at least four monitoring indicators.

More preferably, the list of the monitoring indicators of each status sheet comprises at least nine monitoring indicators.

Advantageously, the list of the monitoring indicators of each status list is identical.

Preferably, the list of the monitoring indicators of each status sheet comprises all or part of the following nine monitoring indicators:
A2. the individual has difficulties in breathing;
A3. the individual is feverish;
A4. the individual has pains;
B2. the individual is not very communicative;
B4. the individual has contacts or visits with his entourage;
C6. the individual is sad;
D2. the individual moves at his home;
D4. the individual prepares his meals;
D7. the individual falls.

Advantageously, the list of the monitoring indicators of each status sheet comprises at least ten monitoring indicators including:
A2. the individual has difficulties in breathing;
A3. the individual is feverish;
A4. the individual has pains;
B2. the individual is not very communicative;
B4. the individual has contacts or visits with his entourage;
C6. the individual is sad;
C7. the individual stores objects in inappropriate locations;
D2. the individual moves at his home;
D4. the individual prepares his meals;
D7. the individual falls.

In particular embodiments of the invention, the data processing system also comprises a device for filling a status sheet.

The device for filling a status sheet may be a portable computer terminal provided with means for communication with the computer server, such as a smartphone or a tablet.

It should be highlighted that, in general, the computer server is not at the home of the individual but located in a remote location. The communication being generally performed via the Internet network and/or the mobile telecommunication network.

In particular embodiments of the invention, the data processing system also comprises at least one sensor transmitting data to a collection terminal configured to process the data and to communicate with the computer server.

To this end, the sensor generally comprises Bluetooth or Wi-Fi type wireless communication means in order to transmit the acquired data.

In general, the collection terminal comprises a microprocessor, a computer memory in order to store the transmitted data and means for communication with the computer server.

Advantageously, the sensor is a sensor for detecting movements.

Such a sensor may be a presence sensor, a camera or an infrared camera.

Thus, depending on the positioning of the sensor(s), all or part of the indicators D1 to D7 could be automatically determined.

In the case where the movement detection sensor is a camera or an infrared camera, a processing of the images is generally performed by the collection terminal.

Advantageously, the sensor is a RFID-type (acronym of “Radio Frequency Identification”) sensor or an NFC-type (acronym of “Near Field Communication”) sensor cooperating with a RFID or NFC tag secured to an object.

As soon as one of the monitored objects is detected as being stored at an unusual location, the indicator C7 automatically takes on the positive value.

In general, the monitored object is an object that is commonly used by the individual such as a pair of slippers, a pair of spectacles, a dental appliance, a hearing aid, a phone or a remote-control.

In particular embodiments of the invention, the sensor is a weight sensor.

Thus, it is possible to estimate the evolution of the weight of the person by detecting an unusual weight.

In particular embodiments of the invention, the data processing system also comprises a database storing the geolocated and dated epidemiological information relating to the temperature of the commune, relating to influenza-like and/or acute diarrhea illnesses.

Thus, it is possible to improve the generation of the parameters of the automatic learning computer algorithm.

According to a second aspect, the invention relates to a data processing method for the prediction of a risk factor of an imminent transfer of an individual to the emergency department.

Such a method comprises a learning phase and an analysis phase.

The learning phase comprises steps of:
acquisition of a plurality of status sheets for each person of a group of persons, each status sheet being dated and including a plurality of monitoring indicators of the corresponding person, each indicator having a value selected in a list of two predetermined values;
acquisition of the dates of transfers of said group of persons to the emergency department;
analysis of the status sheets and of the dates of transfers of all or part of the persons of the group to the emergency department; and
generation of the parameters of an automatic learning computer algorithm from the previous analysis.

The analysis phase comprises steps of:
acquisition of a plurality of status sheets of the individual at distinct time points, each sheet comprising a plurality of monitoring indicators of the individual, each indicator having a value selected in a list of two predetermined values;
determination of a value representative of a risk of an imminent transfer of the individual to the emergency department in the coming days, called risk factor, from the analysis of the evolution of the status sheets of the individual over a predetermined period by the automatic learning computer algorithm whose parameters have been generated during the learning phase;
generation of a warning when the risk factor exceeds a predetermined threshold.

In particular implementations of the invention, the analysis step of the learning phase also takes into account the geolocated epidemiological information.

In particular implementations of the invention, the data processing method also comprises a step of recording the status sheets and the date of transfer of the individual to the emergency department and of updating the parameters of the automatic learning computer algorithm.

The invention also relates to a computer program product implementing the data processing method according to any one of the preceding implementation modes.

### BRIEF DESCRIPTION OF THE FIGURES

Other advantages, objects and particular features of the present invention will come out from the following non-limiting description of at least one particular embodiment of the devices object of the present invention, with reference to the appended drawings, wherein:

FIG. 1 is a simplified diagram of a processing system according to the invention;

FIG. 2 is a flowchart of a processing method implemented by the processing system of FIGS. 1; and

FIGS. 3A-3F are six graphs showing an example of comparison of the predictive results obtained by the method of FIG. 2 according to different combinations of indicators.

### DETAILED DESCRIPTION OF EMBODIMENTS OF THE INVENTION

The present description is provided in a non-limiting way, each feature of one embodiment may be advantageously combined with any other feature of any other embodiment.

As of now, it should be noted that the figures are not to the scale.

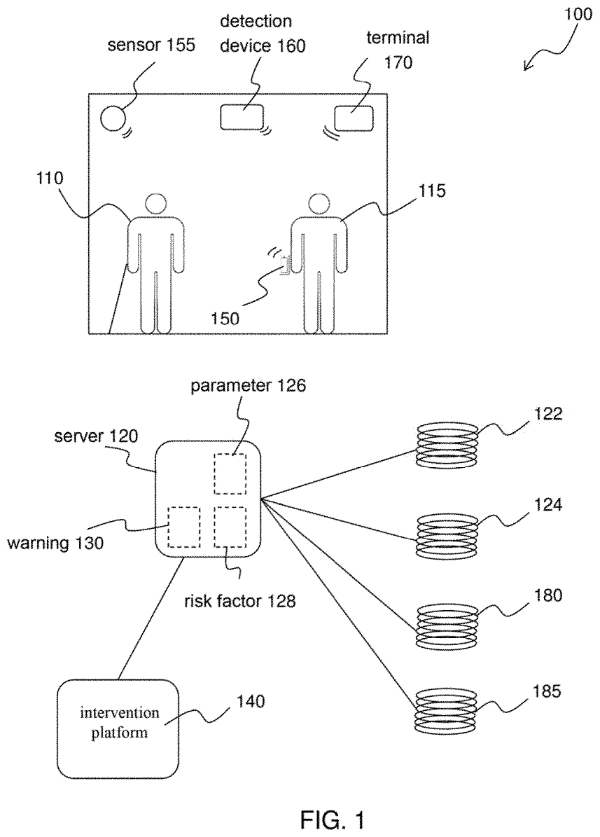

FIG. 1 is a simplified diagram of a data processing system 100 for the determination of a risk factor of an imminent transfer of an individual 110 to the emergency department.

The data processing system 100 comprises a computer server 120 provided with a microprocessor and with a computer memory in which is stored an automatic learning computer algorithm allowing determining a value representative of the risk of the individual 110 being admitted to the emergency department in the near future, corresponding in general to the next seven days. Later on, this value is called risk factor.

It should be highlighted that the automatic learning computer algorithm is generally selected amongst automatic learning techniques, more commonly known under the term “machine learning”, such as a “random forest” type algorithm.

In particular, the determination of the risk factor is performed in a tricky and surprising way by analyzing the evolution of status sheets of the individual 110, each status sheet being established at distinct time points and including a plurality of monitoring indicators of the individual, each indicator having a value selected in a list of two predetermined values, generally a positive value (“Yes”) and a negative value (“No”).

It should be highlighted that the status sheets being in particular devoid of any physiological data of the individual 110, they can be filled by everyone. Thus, for example, each status sheet can be filled by an assistant 115 vising the individual 110. It should be highlighted that a companion of the individual 110 can fill the status sheet instead of the assistant 115.

Each status sheet comprises a list of monitoring indicators generally selected among the following overall list of monitoring indicators:
indicators related to the health condition of the individual, such as:
   A1. the individual has swollen legs;
   A2. the individual has difficulties in breathing;
   A3. the individual is feverish;
   A4. the individual has pains;
relational-type indictors, such as:
   B1. the individual is indifferent;
   B2. the individual is not very communicative;
   B3. the individual lives alone since at least seven days;
   B4. the individual has contacts or visits with his entourage;
behavioral-type indicators, such as:
   C1. the individual refuses toileting assistance;
   C2. the individual does not recognize the companion;
   C3. the individual forgets when the companion has come by;
   C4. the individual communicates inconsistently;
   C5. the individual is aggressive;
   C6. the individual is sad;
   C7. the individual stores objects in inappropriate locations;
   C8. the individual seems tired;
   C9. the individual refuses the intervention of the companion;
indicators representative of the physical and sensory capabilities of the individual, such as:
   D1. the individual stands up;
   D2. the individual moves at his home;
   D3. the individual washes himself;
   D4. the individual prepares his meals;
   D5. the individual leaves his home;
   D6. the individual eats;
   D7. the individual falls.

These indicators may be accompanied with indicators relating to the assistant such as:
E1. the assistant is sad;
E2. the assistant is exhausted.

It should be highlighted that each indicator is representative of a status and that an equivalent formulation of one or several indicator(s) could be used without any notable alteration of the obtained results.

Quite advantageously, the status sheet comprises all or part of the list of the following nine monitoring indicators:
A2. the individual has difficulties in breathing;
A3. the individual is feverish;
A4. the individual has pains;
B2. the individual is not very communicative;
B4. the individual has contacts or visits with his entourage;
C6. the individual is sad;
D2. the individual moves at his home;
D4. the individual prepares his meals; and
D7. the individual falls.

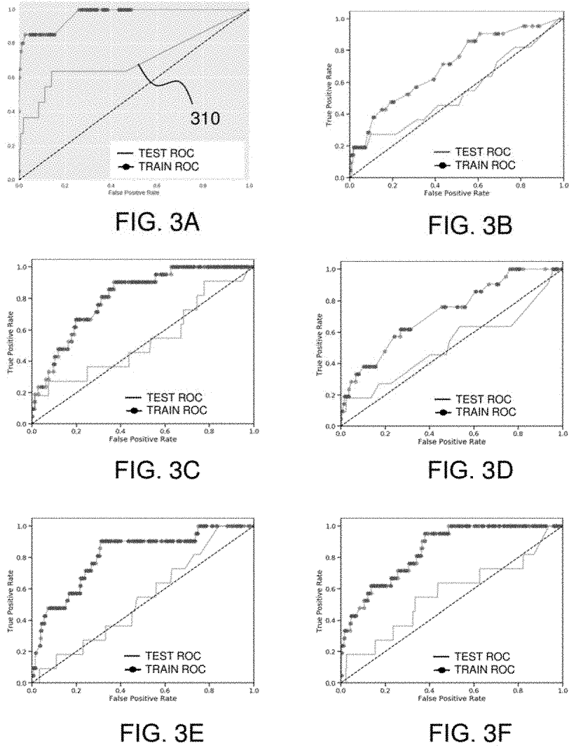

By analyzing the joint evolution of these nine monitoring indicators, it is, quite surprisingly, possible to predict a transfer of the individual 110 to the emergency department in the next seven days with a prediction rate in the range of 70%, which allows obtaining a very rapid support of the individual 110 thereby avoiding his condition getting worse. The predictive performance of this combination of nine indicators is illustrated in FIG. 3A described in more details later on. It should be highlighted that the prediction rate of a transfer to the emergency department in the next fourteen days when taking into account these nine monitoring indicators is in the range of 63%.

In variants of this particular embodiment of the invention, the status sheet comprises the list of the following ten monitoring indicators:
A2. the individual has difficulties in breathing;
A3. the individual is feverish;
A4. the individual has pains;
B2. the individual is not very communicative;
B4. the individual has contacts or visits with his entourage;
C6. the individual is sad;
C7. the individual stores objects in inappropriate locations;
D2. the individual moves at his home;
D4. the individual prepares his meals; and
D7. the individual falls.

In these variants, the indicator C7 has been added with regards to the list comprising nine indicators, which allows improving the prediction of the risk of transfer to the emergency department.

It should be highlighted that the lists the nine or ten monitoring indictors constitute non-limiting examples of the invention and other combinations of at least nine indicators amongst the overall list of monitoring indicators could allow obtaining similar prediction results.

Moreover, in the case where the status sheet only the following four indicators are filled:
A3. the individual is feverish;
B4. the individual has contacts or visits with his entourage;
D2. the individual moves at his home; and
D4. the individual prepares his meals.

The predictive performance of a transfer to the emergency department is in the range of 55%. The predictive performance is similar when the indicator A4 “the individual has pains” is added to this list of four indicators. The predictive performance of this combination of five indicators is illustrated in FIG. 3B.

All or part of the monitoring indicators of the status sheet may be associated to a sub-indicator indicating a precision related to said monitoring indicator, namely an evolution of the status object of said indictor in comparison with the last filling of the status sheet. The sub-indicator is selected amongst three values generally corresponding to an improvement of the status (for example: “better”), to a stabilization of the status (for example: “same”) and to a degradation of the status (for example: “less well”). This sub-indicator allows adding another dimension regarding the indicator whose value has not been modified between two successively filled status sheets.

In general, a sub-indicator is associated to the monitoring indicators A1 to A4, B1, C8, D2, D6 and/or D7.

Thanks to the use of the sub-indicators, it is also possible to improve the prediction of the risk of a transfer of the individual 110 to the emergency department.

The assistant 115, or the companion, may also indicate on the status sheet his general feeling, namely whether the individual 110 is getting better or less well than the last time, or whether his health seems to be identical as the last time.

The computer server 120 is connected to a database 122 storing the status sheets established beforehand for a group of persons and a database 124 storing the dates of transfers of this group of persons to the emergency department.

From the status sheets and the dates of transfers of the group of persons to the emergency department, parameters of the automatic learning computer algorithm are generated by means 126 for generating said parameters. To this end, the computer server 120 may be configured to generate said parameters.

To predict the risk of transfer to the emergency department, the data processing system 100 comprises means 128 for determining the risk factor through the analysis of a plurality of status sheets of the individual 110 thanks to the automatic learning algorithm whose parameters have been generated beforehand.

As soon as the value of the risk factor exceeds a predetermined threshold, a warning is generated by means for generating 130 a warning of the data processing system 100. In particular, this warning may be a text message sent to an intervention platform 140 in order to be able to rapidly take charge of the individual 110.

For the regular filling of the status sheet, the system 100 comprises a device 150 for filling a status sheet which is generally a smartphone or a tablet used by the assistant 115.

Advantageously, the system 100 also comprises, in the present non-limiting example of the invention, at least one sensor 155 for detecting a movement installed at the home of the individual 110 allowing detecting, according to the position of the sensor(s) 155, whether the individual stands up, whether the individual falls, whether the individual moves at his home or whether the individual leaves his home. From the data of the sensor(s), it may also be possible to determine in which room of the home is the individual 110, for example whether he is in a room, a living room, a bathroom or a kitchen.

Thus, all or part of the monitoring indicators D1 to D7 can be automatically determined.

In variants of this particular embodiment of the invention, the system comprises a camera whose data processing allows determining a movement of the individual 110. A face recognition algorithm may also be used to differentiate two individuals.

The system 100 may also comprise a device 160 allowing detecting whether an object is stored at an unusual location. The device 160 may comprise a RFID sensor allowing detecting the presence and/or the position of an object on which a RFID tag is secured.

The monitoring indicator C7 can then be automatically determined.

In variants of this particular embodiment of the invention, the device 160 is based on the combination of sensors and of NFC, instead of RFID, tags.

In order to collect the data originating from the filling device 150, the sensors 155 and/or the detection device 160 and to transmit them to the computer server 120, the system 100 also comprises a collection terminal 170 comprising wireless communication means for receiving these data.

Afterwards, the collection terminal 170 transmits the status sheet filled by the assistant 115, and possibly partially automatically from the data originating from the sensors 155 and/or from the detection device 160, to the computer server 120 which records the status sheet associated to the individual 110 while time-stamping it.

Advantageously, the collection terminal 170 may comprise a clock allowing configuring filling and sending of the status sheet at regular intervals.

It should be highlighted that the status sheet could be filled only partially, with at least the aforementioned nine or ten monitoring indicators, namely the monitoring indicators A2, A3, A4, B2, B4, C6, D2, D4 and D7, and possibly C7. Indeed, the risk factor of a transfer of the individual 110 to the emergency department can be determined based on these nine or ten monitoring indicators.

Once four status sheets have been recorded for the individual 110, the analysis of the evolution of the monitoring indicators can be performed by the automatic learning computer algorithm whose parameters have been generated beforehand.

In order to improve the prediction of a transfer to the emergency department in the near future, the data processing system 100 also comprises a database 180 storing the geolocated and dated epidemiological information relating to the temperature of the commune, relating to influenza-like and/or acute diarrhea illnesses.

By correlating the data of this base 180 with the status sheets of the group of persons and the transfers to the emergency department, it is thus possible to improve the generation of the parameters of the learning computer algorithm, and increase the quality of the prediction of the risk of transfer of the individual 110 to the emergency department.

In order to improve even further the determination of the risk of a transfer to the emergency department, the data processing system 100 may also comprise a database 185 storing an information sheet for each person of the group of persons for which at least one status sheet is stored in the database 122 and/or at least one date of transfer to the emergency department is stored in the database 124. Each information sheet comprising the age of the person, the classification of the person in an iso-resource group (GIR) according to the stage of his loss of autonomy, the assistance plan associated to the person and possibly his medical prescriptions. In general, the assistance plan indicates whether the person needs a homecare attendant, a meals-on-wheels delivery, a housekeeper, and possibly a technical assistance, such as a wheelchair, a cane, a walker or a healthcare bed.

It should be highlighted that the status sheets are generally recorded in the database 122.

Furthermore, as soon as the individual 110 has been transferred to the emergency department, the date of transfer of the individual 110 to the emergency department is recorded in the database 124.

An update of the parameters of the computer algorithm can then be performed while taking into account the date of transfer of the individual 110 to the emergency department.

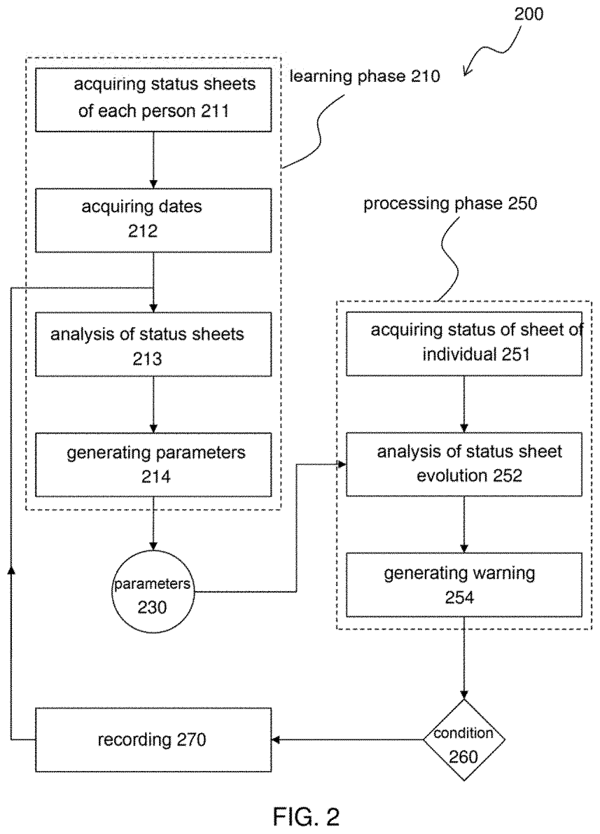

FIG. 2 illustrates, in the form of a flowchart, the data processing method 200 implemented by the data processing system 100.

The data processing method 200 comprises two main phases: a learning phase 210 and a processing phase 250.

The learning phase 210 comprises a first step 211 of acquiring a plurality of status sheets for each person of a group of persons and a second 212 one of acquiring the dates of transfers of the same group of persons to the emergency department.

Afterwards, the status sheets correlated with the dates of transfers of all or part of the persons of the group to the emergency department are analyzed during a third step 213 of the phase 210.

This analysis allows generating the parameters of the automatic learning computer algorithm during a fourth step 214.

In order to improve the determination of the parameters, the analysis performed at step 213 takes also into account, in the present non-limiting example of the invention, the geolocated epidemiological information stored in the database 180.

Afterwards, the parameters generated during step 214 are used during the processing phase 250 which comprises a first step 251 of acquiring a plurality of status sheets of the individual 110.

Afterwards, the evolution of these sheets is analyzed during a second step 252 of the automatic learning computer algorithm whose parameters have been generated during the learning phase 210 in order to determine the value of a risk factor representative of a risk of a transfer to the emergency department in the near future.

When the risk factor exceeds a predetermined threshold, a warning is generated during a fourth step 254.

If the individual 110 is admitted to the emergency department, represented by the condition 260, the method 200 may advantageously update the parameters of the automatic learning computer algorithm.

To this end, the method 200 comprises a step 270 of recording the status sheets and the date of transfer of the individual 110 to the emergency department respectively in the database 122 and 124.

Steps 213 and 214 of the learning phase are then performed again to update the parameters of the computer algorithm.

FIGS. 3A-3F represent an example of comparison of the results obtained by basing the analysis on different combinations of indicators.

In other words, when an analysis is based on a determined combination of indicators, the steps of analyzing 213 and generating 214 the parameters of the algorithm executed during the learning phase 210 are performed while considering only the indicators of this combination on the status sheets. If the status sheet comprises other indicators, these are not considered, which amounts to the status sheet comprising only the determined combination of indicators.

The step of determining the value of the risk factor through the analysis 252 of the status sheets of the individual 110 executed during the processing phase 250 is also performed while considering only the indicators of the determined combination.

FIGS. 3A-3F are six graphs, each corresponding to a combination of indicators.

Each graph comprises two curves ROC (acronym of “Receiver Operating Characteristic”) allowing characterizing the performance of a binary classifier by representing the true positive rate, that is to say the fraction of positives that are effectively detected, as a function of the false positive rate, fraction of the negatives that are wrongly detected.

In each graph, the true positive rate, indicated in FIGS. 3A-3F by the term “True Positive Rate”, is in the ordinates, whereas the false positive rate, indicated in FIGS. 3A-3F by the term “False Positive Rate”, is in the abscissas.

Moreover, the curve “TRAIN ROC” illustrates the learning phase 210 during which the parameters of the algorithm according to the analysis of the status sheets stored in the database 122.

In turn, the curve “TEST ROC” illustrates the processing phase 250 during which a value representative of a risk of a transfer of the individual 110 to the emergency department is calculated. To establish the curve, this analysis is performed on a plurality of individuals 110, selected randomly, in order to calculate the predictive performance represented by the surface located under the curve “TEST ROC” by comparing the obtained value of the risk factor with the actual transfer to the emergency department stored in the database 124.

To this end, the curves “TRAIN ROC” and “TEST ROC” have been calculated in the present example, by defining two cohorts from the actual data stored in the database 124. The first cohort, corresponding to 70% of the persons registered in the databases 122 and 124, is used to establish the curve “TRAIN ROC”. Whereas the second cohort, corresponding to 30% of the persons registered in the databases 122 and 124, is used to establish the curve “TEST ROC”.

The graph represented in FIG. 3A corresponds to the combination of the nine indicators:
A2. the individual has difficulties in breathing;
A3. the individual is feverish;
A4. the individual has pains;
B2. the individual is not very communicative;
B4. the individual has contacts or visits with his entourage;
C6. the individual is sad;
D2. the individual moves at his home;
D4. the individual prepares his meals; and
D7. the individual falls.

It should be highlighted that the slope at the origin of the curve 310, corresponding to the curve “TEST ROC” of FIG. 3A, is almost vertical, which sets out an advantage of the use of this combination of nine indicators in the data processing method 200 for the determination of the risk factor of an imminent transfer to the emergency department. Indeed, this vertical slope indicates that the transfer of most of the first individuals 100 object of the analysis to the emergency department will be effectively detected. Thus, they can be taken in charge very rapidly by a healthcare service.

The graph represented in FIG. 3B corresponds to the combination of the five indicators:
A3. the individual is feverish;
A4. the individual has pains;
B4. the individual has contacts or visits with his entourage;
D2. the individual moves at his home; and
D4. the individual prepares his meals.

The obtained predictive performance is in the range of 54%, with a slope that is also vertical at the origin.

The graph represented in FIG. 3C corresponds to the combination of the nine indicators:
A4. the individual has pains;
B1. the individual is indifferent;
B2. the individual is not very communicative;
C1. the individual refuses toileting assistance;
C2. the individual does not recognize the companion;
C6. the individual is sad;
D7. the individual falls;
E1. the assistant is sad; and
E2. the assistant is exhausted.

The predictive performance obtained with this combination is 53%.

The graph represented in FIG. 3D corresponds to the combination of the eight indicators:
A1. the individual has swollen legs;
A2. the individual has difficulties in breathing;
A3. the individual is feverish;
B2. the individual is not very communicative;
B4. the individual has contacts or visits with his entourage;
D2. the individual moves at his home;
D4. the individual prepares his meals; and
D7. the individual falls.

The predictive performance obtained with this combination is 52%.

The graph represented in FIG. 3E corresponds to the combination of the eight indicators:
A2. the individual has difficulties in breathing;
A4. the individual has pains;
C1. the individual refuses toileting assistance;
C6. the individual is sad;
C8. the individual seems tired;
D2. the individual moves at his home;
D4. the individual prepares his meals; and
D7. the individual falls.

The predictive performance obtained when basing the analysis on this combination is 53%.

The graph represented in FIG. 3F corresponds to the combination of the six indicators:
A1. the individual has swollen legs;
A2. the individual has difficulties in breathing;
A3. the individual is feverish;
A4. the individual has pains;
B3. the individual lives alone since at least seven days; and
D7. the individual falls.

The predictive performance obtained when basing the analysis on this combination is 56%.

1-14. (canceled)

15. A data processing system to determine a risk factor of an imminent transfer of an individual to an emergency department, comprising:
a computer server comprising a microprocessor and a computer memory;
a first database to store a plurality of status sheets for each person of a group of people;
a second database to store dates of transfers of said group of people to the emergency department, each status sheet being dated and comprising a list of monitoring indicators, each monitoring indicator having a value selected from a list of two predetermined values according to a status of a corresponding person;
a portable device to fill a status sheet of the individual; and
the microprocessor configured to:
generate parameters of an automatic learning computer algorithm from the status sheets and the dates of transfers of the group of people to the emergency department;
determine the risk factor by analysis of a plurality of status sheets of the individual using the automatic learning algorithm whose parameters have been previously generated, each status sheet of the individual being established at distinct time points; and
generate a warning when the risk factor exceeds a predetermined threshold.

16. The data processing system of claim 15, wherein the portable device is a smartphone or a tablet.

17. The data processing system of claim 15, wherein the monitoring indicators are selected among:
indicators related to a health condition of the individual, comprising:
A1: the individual has swollen legs;
A2: the individual has difficulties in breathing;
A3. the individual is feverish; and
A4. the individual has pains;
relational-type indictors, comprising:
B1. the individual is indifferent;
B2. the individual is not very communicative;
B3. the individual lives alone since at least seven days; and
B4. the individual has contacts or visits with his entourage;
behavioral-type indicators, comprising:
C1. the individual refuses toileting assistance;
C2. the individual does not recognize the companion;
C3. the individual forgets when the companion has come by;
C4. the individual communicates inconsistently;
C5. the individual is aggressive;
C6. the individual is sad;
C7. the individual stores objects in inappropriate locations;
C8. the individual seems tired; and
C9. the individual refuses the intervention of the companion; and
indicators representative of physical and sensory capabilities of the individual, comprising:
D1. the individual stands up;
D2. the individual moves at his home;
D3. the individual washes himself;
D4. the individual prepares his meals;
D5. the individual leaves his home;
D6. the individual eats; and
D7. the individual falls.

18. The data processing system of claim 17, wherein all or part of the monitoring indicators are associated with a sub-indicator indicating a precision relating to said monitoring indicators with respect to a last filling of the status sheet, the sub-indicator being selected amongst three values.

19. The data processing system of claim 15, wherein the list of the monitoring indicators comprises at least nine monitoring indicators.

20. The data processing system of claim 15, wherein the list of the monitoring indicators comprises all or part of the following nine monitoring indicators:
A2. the individual has difficulties in breathing;
A3. the individual is feverish;
A4. the individual has pains;
B2. the individual is not very communicative;
B4. the individual has contacts or visits with his entourage;
C6. the individual is sad;
D2. the individual moves at his home;
D4. the individual prepares his meals; and
D7. the individual falls.

21. The data processing system of claim 15, wherein the list of the monitoring indicators comprises at least following ten monitoring indicators:
A2. the individual has difficulties in breathing;
A3. the individual is feverish;
A4. the individual has pains;
B2. the individual is not very communicative;
B4. the individual has contacts or visits with his entourage;
C6. the individual is sad;
C7. the individual stores objects in inappropriate locations;
D2. the individual moves at his home;
D4. the individual prepares his meals; and
D7. the individual falls.

22. The data processing system of claim 15, comprising at least one sensor transmitting data to a collection terminal configured to process the data and to communicate with the computer server.

23. The data processing system of claim 22, wherein said at least one sensor is a sensor configured to detect a movement.

24. The data processing system of claim 22, wherein said at least one sensor is a RFID sensor or an NFC sensor respectively cooperating with a RFID or NFC tag secured to an object.

25. The data processing system of claim 22, wherein said at least one sensor is a weight sensor.

26. The data processing system of claim 15, comprising a third database to store geolocated and dated epidemiological information relating at least one of the following: a temperature of the commune, influenza and acute diarrhea illnesses.

27. A data processing method for determining a risk factor of an imminent transfer of an individual to an emergency department, implemented by the processing system of claim 15, the method comprising:
a learning phase comprising:
acquisition of said plurality of status sheets for said each person of the group of people;
acquisition of the dates of transfers of said group of people to the emergency department;
analysis of said plurality of status sheets and the dates of transfers of all or part of the people of the group to the emergency department;
generation of the parameters of the automatic learning computer algorithm from a previous analysis;
a processing phase comprising:
acquisition of the plurality of status sheets of the individual at distinct time points;
determination of a value representative of a risk of an imminent transfer of the individual to the emergency department in coming days from the analysis of an evolution of the plurality of status sheets of the individual over a predetermined period by the automatic learning computer algorithm whose parameters have been generated during the learning phase; and
generation of the warning when the risk factor exceeds the predetermined threshold.

28. The method of claim 27, wherein the analysis of the learning phase considers geolocated epidemiological information.

29. The method of claim 27, further comprising recordation of the plurality of status sheets and the date of transfer of the individual to the emergency department and update of the parameters of the automatic learning computer algorithm.
